(19) United States
(12) Patent Application Publication
(10) Pub. No.: US 2016/0078780 A1
(43) Pub. Date: Mar. 17, 2016

(54) Method and System for Aerobic and Cognitive Training

(71) Applicant: The Arizona Board of Regents for and on behalf of the University of Arizona, Tucson, AZ (US)

(72) Inventors: Gene Evans Alexander, Tucson, AZ (US); David Allan Raichlen, Tucson, AZ (US)

(73) Assignee: The Arizona Board of Regents for and on behalf of the University of Arizona, Tucson, AZ (US)

(21) Appl. No.: 14/855,127

(22) Filed: Sep. 15, 2015

Related U.S. Application Data
(60) Provisional application No. 62/050,679, filed on Sep. 15, 2014.

(51) Int. Cl. G09B 19/00; A61B 5/11; A61B 5/00; A61B 5/021

(52) U.S. Cl. CPC G09B 19/00; A61B 5/021; A61B 5/1118; A61B 5/486; A61B 5/4884

(57) ABSTRACT

A method for cognitive training includes administering a complex spatial navigation task to a subject. Additional cognitive tasks may be administered before, during, and/or after the complex spatial navigation task. In some embodiments, the subject engages in physical activity during the complex spatial navigation task.

### BACKGROUND

Cognitive decline may be caused by a variety of things, from aging to stress to injury to disease. However, much like physical conditioning can improve the performance of an athlete, the brain can also be trained to improve performance. Given that the brain has great potential for neuroplasticity, training can provide ways to enhance brain function and cognition over the lifespan. Such training has the potential to improve cognitive performance during healthy aging as well as in the context of neurological and psychiatric disorders like Alzheimer's disease, cerebrovascular disease, Parkinson's disease, multiple sclerosis, traumatic brain injury, developmental disorders, attention deficit hyperactivity disorder, and other mental health conditions.

### SUMMARY OF THE INVENTION

The present invention provides a method and system for aerobic and cognitive training, and an apparatus for monitoring subject response during the same. In accordance with one embodiment of the invention, the method includes administering a complex spatial navigation task to a subject. Preferably, this task is administered while the subject is participating in physical activity. In some embodiments, one or more additional cognitive tasks may be administered to the subject during the complex spatial navigation task. The one or more additional cognitive tasks is preferably selected from a group of cognitive tasks that test memory, executive functions, information processing speed, language processing, and visuospatial/visuoperceptual functions.

In accordance with one embodiment of the present invention, the system includes a sensor for monitoring a subject's physiological response to physical activity; a first input device for providing a response from a subject in response to a spatial navigation activity; a second input device for providing a response from a subject in response to a simultaneously administered cognitive task; and a display device connected to or integral with a computing device including a non-transitory computer readable storage medium storing software configured to administer a complex spatial navigation task to a subject on the display device and to administer a series of cognitive tasks to the subject during the course of the spatial navigation task. The computing device receives responses to the spatial navigation task and the series of cognitive tasks from the first and second input devices. The software tracks, for example, the number of turns made during the spatial navigation task, the overall time to completion of the navigation challenge, and the number of errors in going outside the guidelines of a navigation path. The software also determines at least one of accuracy or response time for each of the cognitive tasks administered, stores the accuracy and/or response time, and provides feedback to the subject regarding subject's performance. In some embodiments, the software is configured to monitor data collected from the one or more sensors regarding subject's physical response to activity, and prompt the subject to stay within defined parameters for level of exertion.

### BRIEF DESCRIPTION OF THE DRAWINGS

FIG. 1 is a block diagram of a system for cognitive training in accordance with the present invention.

FIG. 2 is an exemplary illustration of the platform displayed to the subject, in accordance with the present invention.

FIG. 3 is an exemplary illustration of the platform shown in FIG. 2, where an executive function inhibition task is presented during spatial navigation, in accordance with the present invention.

FIG. 4 is an exemplary illustration of the platform shown in FIG. 2, where a simple or choice reaction time information processing speed task is presented during spatial navigation, in accordance with the present invention.

FIG. 5 is an exemplary illustration of the platform shown in FIG. 2, where a verbal-paired associates memory task is presented during spatial navigation, in accordance with the present invention.

FIG. 6 is an exemplary illustration of the platform shown in FIG. 2, where an executive function number-letter switching task is presented during spatial navigation, in accordance with the present invention.

FIG. 7 is an exemplary illustration of the platform shown in FIG. 2, where an executive function updating n-back task is presented during spatial navigation, in accordance with the present invention.

FIG. 8 is an exemplary illustration of the platform shown in FIG. 2, where a verbal list-learning memory task is presented during spatial navigation, in accordance with the present invention.

FIG. 9 is an exemplary feedback device in accordance with the present invention.

FIG. 10 illustrates exemplary positioning of the feedback device shown in FIG. 9, in accordance with the present invention.

FIG. 11 illustrates an exemplary implementation of the present invention, using a recumbent stationary bicycle.

FIGS. 12 and 13 illustrate exemplary screenshots of Module 1.

FIGS. 14-17 illustrate exemplary screenshots of the verbal paired-associates task of Module 2.

FIGS. 18 and 19 provide exemplary screenshots of inhibition tasks in accordance with Module 3.

FIGS. 20 and 21 provide exemplary screenshots of switching tasks in accordance with Module 3.

FIGS. 22 and 23 provide exemplary screenshots an updating/monitoring task in accordance with Module 3.

FIGS. 24 and 25 provide exemplary screenshots of the simple reaction time task in accordance with Module 4.

FIG. 26 provides an exemplary screenshot of the choice reaction time task in accordance with Module 4.

FIG. 27 provides an exemplary screenshot of the complex spatial navigation task in accordance with Module 5.

### DETAILED DESCRIPTION

Reference throughout the specification to “one embodiment,” “another embodiment,” “an embodiment,” “some embodiments,” and so forth, means that a particular element (e.g., feature, structure, property, or characteristic) described in connection with the embodiment is included in at least one embodiment described herein, and may or may not be present in other embodiments. In addition, it is to be understood that the element(s) described herein may be combined in any suitable manner in the various embodiments.

A method for cognitive training, in accordance with the present invention, includes administering a complex spatial navigation task to a subject. A complex spatial navigation task is a spatial navigation task that requires that the subject navigate between two or more points while also staying within the bounds of a prescribed path, such as a road, sidewalk, bike path, or the like. A complex spatial navigation task may optionally include a map or other navigation aid, such as the use of landmarks, global positioning system (GPS) coordinates, or directions, to aid the subject in navigation between the two or more points. When a complex spatial navigation task including a navigation aid is repeated, the complex spatial navigation task also includes additional learning components as the test subject is repeatedly exposed to the same landmarks, coordinates, cues, spatial navigation procedures, etc. When additional learning components are included, the subject's performance with respect to these learning components may also be assessed. In some embodiments, one or more additional cognitive tasks are administered to the subject before, after, and/or while the subject completes a complex spatial navigation task.

The difficulty or complexity of both the complex spatial navigation task and additional cognitive tasks may be varied. With respect to the complex spatial navigation task, the difficulty may be increased, for example, by increasing the number of decision points (points where the subject is expected to make a decision regarding direction of travel), increasing the number of options for direction of travel at decision points, and increasing the length or duration of the complex spatial navigation task, or any combination thereof. The difficulty of the complex spatial navigation task may also be decreased when appropriate for the test subject—by reducing the number of decision points, the number of options for direction of travel at decision points, and decreasing the length or duration of the complex spatial navigation task, or any combination thereof. The difficulty of other cognitive tasks may be altered by increasing/decreasing the number of stimuli presented or their complexity and/or increasing/decreasing the number of cycles of stimuli presented to the subject.

Preferably, the complex spatial navigation task and additional cognitive tasks are completed while the subject is engaged in physical activity. For example, the subject may be engaged in an aerobic activity, such as walking, jogging, cycling, or using cardio equipment such as a stationary bike, step machine, treadmill, elliptical machine, or the like. In some implementations, the subject is provided with a sensor to monitor the subject's level of exertion during cognitive training. Further, the subject may wear or use a heart rate monitor during administration of the tasks, and be prompted to keep his or her heart rate within a specified range, for example between 60% and 80% of the subject's age-adjusted maximum heart rate. In still other implementations, the subject may be fitted with a sensor to monitor activity, such as an accelerometer. In some implementations, the accelerometer data may be correlated with the heart rate monitor data. Either alone, or in combination, these sensors may determine speed of movement through the spatial navigation task.

In one embodiment, the system will be calibrated such that the speed of progression in the virtual scene is determined by the subject's physical exertion as indicated by heart rate monitor data and/or accelerometer data. The subject may be prompted to increase or decrease effort based on the calibration and real-time sensor monitoring. In some embodiments, subject effort may be gauged using one or more sensors such as a heart rate monitor, one or more galvanic skin response sensors, or by monitoring electrical activity in the subject, for example, by surface electrodes. These sensors, alone or in combination, can be used to gauge aerobic intensity, and to provide feedback so that a subject stays within a target aerobic intensity range. In yet another embodiment, a piece of exercise equipment used by the subject may be connected with the system of the present invention, for example via a wired or wireless peripheral connection to a computer system, so that speed of movement and/or resistance can be altered by the system based on subject effort. Based on sensor input, the speed and/or resistance of the cardio machine may be altered (increased or decreased) to allow subjects to exercise in target “zones,” which are defined by subject exertion measured by one or more sensors as described above. These zones may be pre-defined, or defined for each subject. For example, the speed and/or resistance of the exercise equipment may be altered so that the subject's heart rate is maintained between 60% and 80% of that subject's maximum heart rate. Similarly, a subject that is recovering from a cardiac event may be instructed by a health care professional to limit his or her activity and, thus, the system may be programmed to limit the speed and/or resistance of the exercise equipment so that the subject's heart rate does not exceed 50% of the subject's maximum heart rate.

It should be appreciated that the methods described herein may be performed using instructions encoded by one or more computer programs comprising code portions which, when stored on a non-transitory computer-readable storage medium and loaded and run on a computing device, cause the computer to execute any one of the methods defined herein.

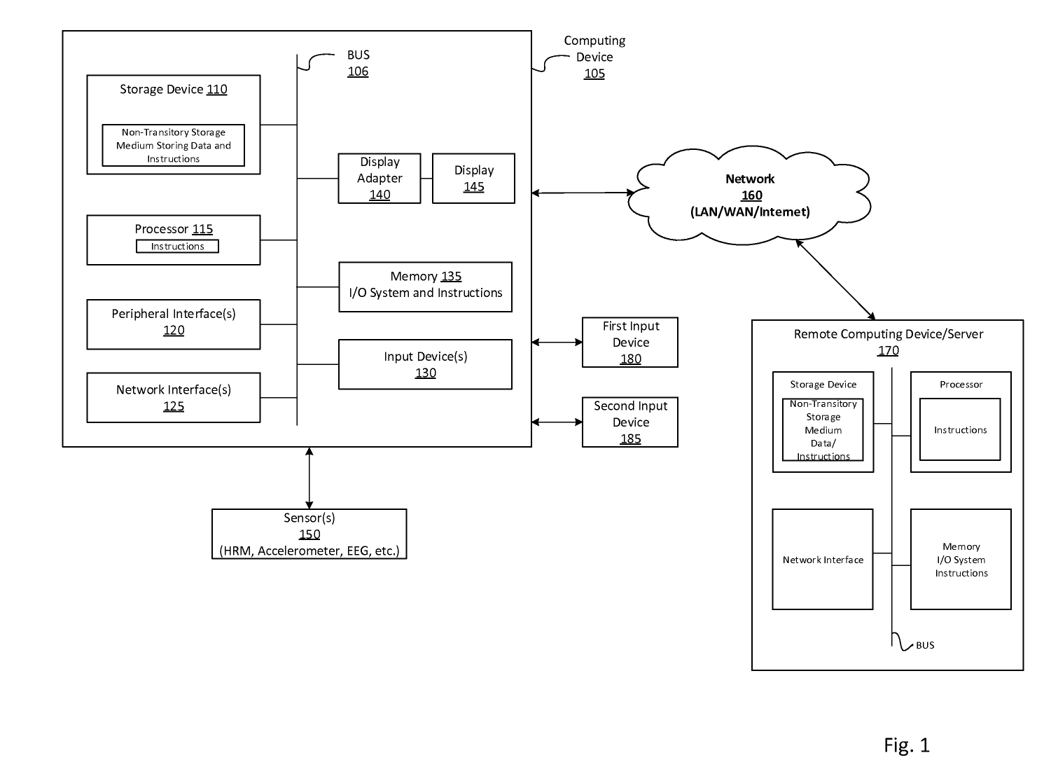

An exemplary system for administering the method of the present invention is described in FIG. 1. The system includes a computing device 105 that receives input from a first input device 180, a second input device 185, and one or more sensors 150. Computing device 105 may communicate data, including subject information and/or results, to a remote computing device/server 170 via a wired or wireless network connection 160. Computing device 105 may be any computing device including, but not limited to, a smartphone, a “phablet”, a tablet computing device, a notebook/laptop computer, a desktop computer, or a computer that is integrated with exercise equipment. Computing device 105 includes a storage device 110 configured to store both software configured to implement the method of the present invention and data acquired during administration of the method of the present invention on a non-transitory, computer-readable storage medium, such as a hard drive, hybrid drive, flash memory or the like. Computing device 105 also includes system memory 135 and a processor to execute the instructions provided in the stored software. Computing device 105 may be connected to one or more remote computing devices 170 via a wired or wireless (wi-fi, Bluetooth, or the like) network interface 125 using any known networking protocol. Computing device 105 also includes a display adapter 140 configured to display the output of software running on computing device 105. The output may be displayed on a display 145 that is integral with computing device 105, for example, the display of a laptop/notebook computer, phone, or tablet, or any display that may be directly connected with computing device 105, such as a monitor (LCD, LED, etc.) or television, or a remote display using, for example, streaming technologies such as Apple TV, Roku, Google Chromecast, or any other display technology including virtual reality displays. Further, one or more peripherals such as microphones and speakers or headphones (wired or wireless) may be connected with the system via peripheral interface 420 to facilitate the use of audio input/output.

Computing device 105 is configured to receive inputs from a first input device 180, a second input device 185 and, optionally, one or more sensors 150 for measuring subject activity and/or exertion. Each of the first input device, the second input device, and the one or more sensors may be connected to computing device 105 via a wired connection, such as a universal serial bus (USB) or serial port connection, or a wireless connection, such as a Bluetooth, Zigbee, wi-fi, ANT+, or any other wireless protocol. The first input device is preferably configured so that a subject action, such as applying pressure or pressing a button, indicates a choice of direction by the subject. The first input device may be located in a central position for the subject (for example, a joystick), or may be configured such that one or more directional options are presented on each side of the subject's body (for example, controls in both the right and left hands). The second input device is preferably configured so that a subject action indicates a positive response and a lack of subject action indicates a negative response. In some implementations, the second input device is configured to allow a subject to indicate positive responses and negative responses on both the left side of the body and the right side of the body (for example controls for both the right and left hands). In still other implementations, the first and second input devices may be consolidated into a single feedback device, where the single feedback device provides feedback from both the left side of the subject and the right side of the subject. Sensor(s) 150 include any sensors that may measure subject activity and/or exertion including, but not limited to a pressure sensor, a single or multi-axis accelerometer, a heart rate monitor, galvanic skin response sensors, or the like. Further, it should be appreciated that, when a subject is not able to provide a motor response to stimuli, sensors may be configured to receive another type of signal or sound as a response from a subject, for example, sensors may be configured to detect/receive electrical signals from the body of the subject via electroencephalography (EEG) or the like.

A complex spatial navigation task requires a subject to navigate between two points, for example through a prescribed course, while staying within the bounds of a defined path. In an exemplary implementation, the complex spatial navigation task requires the subject to navigate through a maze, which may be presented to the subject in two dimensions on a display device such as a screen, or in three dimensions presented via an augmented reality or virtual reality system such as Oculus Rift, Avegent Glyph, Google Cardboard, or the like. The complex spatial navigation task, however, is not limited to presenting a maze and may be any spatial navigation task that requires multi-tasking, such as using a map to navigate a physical or virtual course, or following clues or prompts to virtually or physically navigate between two or more points. For example, a subject may be prompted to navigate between two points on a map, navigate through a room, a structure, a town, or navigate through a presented layout.

In another implementation, another visuospatial task may be substituted for the spatial navigation task. In a non-limiting example, the subject may move through a scene without navigating between points, for example by following a prescribed path. In this case, a subject would be scored based on the subject's ability to stay on the path. In another non-limiting example, a subject may be tasked with locating one or more objects in a “virtual” room or scene.

In some implementations, while the subject is navigating the maze, the subject is presented with one or more additional cognitive tasks that assess and train different cognitive domains. These cognitive domains may include, for example, memory, including but not limited to episodic memory, associative memory, and spatial memory; executive functions, information processing speed, language processing, and visuospatial/visuoperceptual functions. Exemplary cognitive tasks may include, but are not limited to, recall of route, recall of landmarks associated with objects displayed during the task, recall of objects presented on road signs during the task, inhibition of position of directional arrow signs on the screen during navigation, switching between cognitive sets for judgments about numbers and letters, and recall of presented integers with sequential updating to assess and train executive working memory. It should be appreciated that the series of cognitive tasks may be presented in any order, but some orders are preferred under some conditions as in the exemplary battery provided herein.

In some implementations, at least a portion of one or more cognitive tasks may be implemented using auditory task stimuli, cues, prompts, or instructions. For example, a subject may be prompted to follow auditory prompts to navigate a spatial navigation task, or may be instructed to press a series of buttons in a specific order as part of the series of cognitive tasks. It should be appreciated that a computing device may provide audio output in some implementations, and that this output may be conveyed to the subject via wired or wireless headphones, one or more speakers, or may be read to the subject by a training administrator without departing from the scope of the invention described herein.

A subject's cognitive performance is evaluated based on accuracy of response and response time. This evaluation may include consideration of parameters including, but not limited to, the number of complex spatial navigation tasks completed, the time required for completion of each complex spatial navigation task and each additional cognitive task administered, the number of times a subject turns during the complex spatial navigation task, the number of turns required to solve the complex spatial navigation task, the number of “correct” turns, the number of turns made in error, subject accuracy, time on path, time in the center of the path, center width, center accuracy, distance traveled by subject, the travel distance necessary to solve the complex spatial navigation task, etc. Further, a subject's physical performance can be tracked using a sensor such as a heart rate monitor. Feedback regarding cognitive performance and/or physical performance may be provided in real time via audio or visual feedback, and/or may stored, for example in a local or remote computer database, and monitored over time.

The method described herein can then be adapted to vary the level of cognitive challenge presented to the subject based on the performance of that specific subject. Generally, the level of cognitive challenge will increase over time, for example, by introducing new and varied complex spatial navigation tasks having more decision points, more options for directions of travel at one or more decision points, and/or increasing the duration or length of the complex spatial navigation task. The difficulty of any additional cognitive tasks administered may also be increased as appropriate for the specific cognitive task being administered. However, it should be appreciated that the level of cognitive challenge may also be decreased, for example, to minimize the level of frustration of a subject so that the subject continues to complete the tasks presented, thus encouraging compliance with a prescribed regimen. Complex spatial navigation tasks may be simplified by, for example, reducing the number of decision points, reducing options for direction of travel at one or more decision points, and/or decreasing the duration or length of the complex spatial navigation task. The difficulty of any additional cognitive tasks administered may also be decreased as appropriate for the specific cognitive task being administered. The adaptable nature of this method makes it suitable for subjects having a wide range of cognitive abilities. For example, the method can be adapted to work across the lifespan; for individuals at risk for developing and for those experiencing neurodegenerative diseases such as Alzheimer's and cerebrovascular disease, multiple sclerosis, and Parkinson's disease; and for those with traumatic brain injuries. In addition, the method can be adapted to work for individuals at risk for or with psychiatric conditions, such as attention deficit hyperactivity disorder, developmental disorders, or other mental health conditions. Further, while specific numbers of tasks and intervals for tasks are described herein, it should be appreciated that the number of tasks, intervals for tasks, and the timing of administration of task trials may be varied without departing from the scope of the invention.

In some implementations, milestones may be created, whether for a “category” of subject based on cognitive ability or cognitive impairment, or for an individual subject. A subject's progress toward a milestone may be displayed, for example, on the display device. In some implementations, when the subject is able to communicate with other subjects via, for example, the Internet, the subject may be able to track his or her progress against peers and “compete” against other subjects to serve as a motivational tool for completing the tasks described herein. Similarly, a subject's performance can be tracked remotely by a health care professional.

A subject's performance may also serve as a diagnostic tool by using specific spatial navigation tasks combined with a specific, prescribed series of cognitive tasks such as memory, executive function, information processing speed, language processing, and visuospatial/visuoperceptual tasks that form a standardized assessment. Performance on these standardized assessments, as opposed to the subject-specific adapted assessments, can be compared to other subjects using known statistical analysis methods. By administering a standardized assessment, the method and system described herein may also be used as a diagnostic tool. Data from known populations, such as individuals with diagnosed neurological disorders, may be used to establish ranges of scores typical for an individual having a specific neurological disorder or risk for such disorder. The performance of a subject on a standardized assessment may be compared to the performance of other subjects in order to serve as a diagnostic tool or test for use in diagnosing neurological or psychiatric disorders or risk for a disorder, such as Alzheimer's disease, cerebrovascular disease, Parkinson's disease, multiple sclerosis, traumatic brain injury, developmental disorders, attention deficit/hyperactivity disorder, and the like. If a subject is diagnosed with a specific neurological or psychiatric disorder or is identified as being at risk for a specific neurological or psychiatric disorder, a cognitive training plan may be created and tailored for that subject to improve the subject's cognition and potentially improve the subject's quality of life by reducing the effects of the disease. By tailoring the cognitive training plan, a subject's plan may focus, for example, on executive function tasks if executive function is known to be compromised by the subject's diagnosed disease (or risk factors for the disease). Such a cognitive training plan can also be used in combination with other specified treatments or prevention therapies, for example in the case of a pharmacological treatment for Alzheimer's disease, to enhance the benefit of the other treatments or interventions.

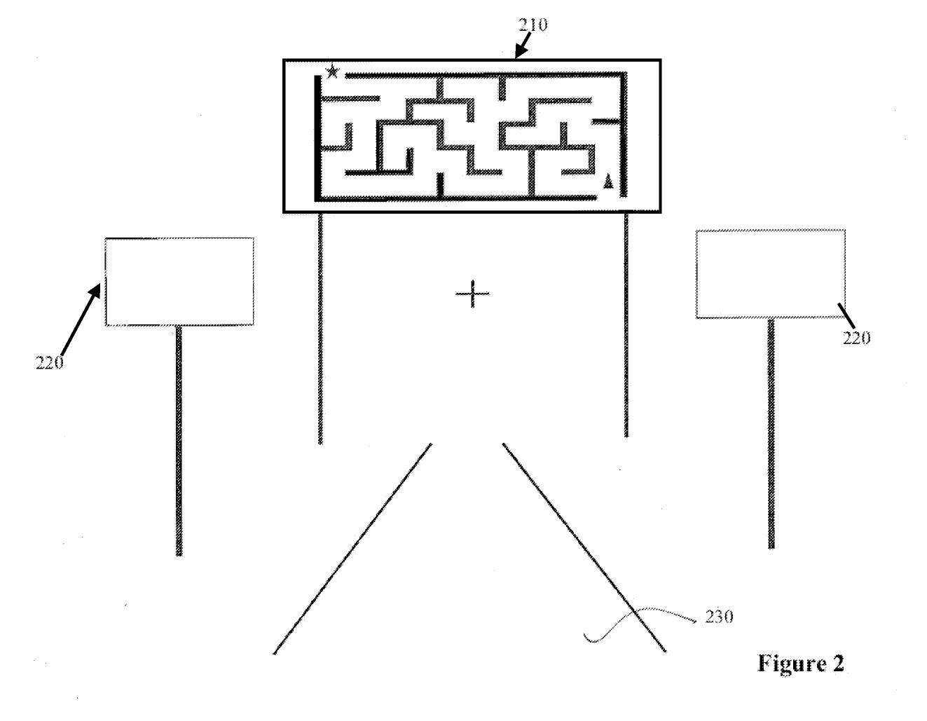

FIG. 2 is an exemplary illustration of the platform displayed to the subject, in accordance with the present invention, when tasks are displayed to the subject in a two-dimensional environment, such as a computer screen. The spatial navigation task is presented to the subject at the top center of the complex spatial navigation task display 210, and the subject's position in the maze is displayed, for example, as an arrowhead. A path 230 is provided for the subject to follow, and is displayed at the bottom center of the complex spatial navigation task display 210. In some implementations, the path may be displayed with scenery surrounding the path. In some implementations, a sensor worn by the subject, such as an accelerometer or heart rate monitor, may be used to progress the subject through the maze based on the subject's movement or exertion. For example, where changes in heart rate within the target zone determine a graded speed response in the spatial navigation task. In other implementations, for example, where a subject is on a piece of cardio equipment, the speed determined by the cardio equipment may be used to progress the subject through the spatial navigation task. The subject may indicate a turn, for example in 90 degree increments, by depressing a switch on a feedback device. As used herein, a switch refers to any device that indicates subject response and includes, but is not limited to, a pressure sensor, a two position switch, a depressible button, a multi-position switch, or any other mechanism that can indicate a binary (yes or no) response. In other implementations, a turn may be indicated, for example, by turning handlebars on a stationary bike. In still other implementations, a pressure sensor may allow the subject to provide a graded response, for example, the degree of the turn will increase as the subject applies increasing pressure to the pressure sensor. In some implementations, such as when a subject's motor function is compromised, a microphone may be used to record subject response communicated via sound. The star at the end of the spatial navigation task indicates the “finish.”

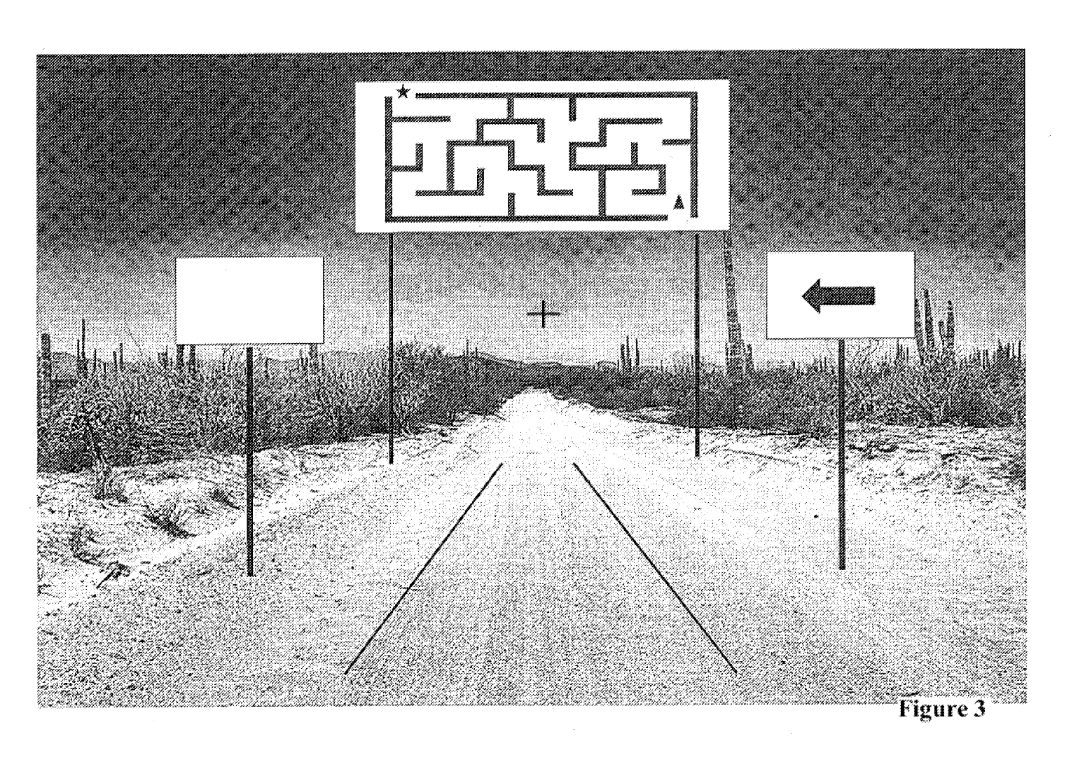

One or more additional cognitive tasks may be administered during the complex spatial navigation task via the smaller placards 220 flanking the complex spatial navigation task display 210 in FIG. 2. These placards will collectively be referred to herein as the “cognitive task display area.” These are shown in more detail in FIGS. 3, 4, 5, 6, 7, and 8. While 2 placards are illustrated in the examples presented herein, it should be understood that the number, size, appearance, and position of the placards may be varied without departing from the scope of the present invention. Similarly, the input/feedback devices described herein may be adapted to receive input regarding the various cognitive tasks presented to the subject.

While a specific arrangement is described with respect to the spatial navigation task display and the cognitive task display area, it should be appreciated that this arrangement is only exemplary and is non-limiting. For example, in some implementations, the spatial navigation task display area may be presented to one side or the other, and the cognitive task display area may be centered. In still other implementations, the spatial navigation task may be presented at the center of the display area, and the series of cognitive tasks may be presented at the periphery of the display area in any direction. In other implementations, the spatial navigation task may be hidden from view for all or part of the training, for example, the spatial navigation task may be hidden from view while the subject is completing a task in the series of cognitive tasks. In still other implementations, the complex spatial navigation task may be presented via a virtual or augmented reality device.

FIG. 3 is an exemplary illustration of the platform shown in FIG. 2, where an executive function inhibition task is presented during the complex spatial navigation task, in accordance with the present invention. Note that a scenic background is provided for the task, but any background may be provided for the task. In the executive function inhibition task, the cognitive task display area displays a directional arrow, and the subject is tasked with indicating the appropriate direction via a feedback mechanism, such as a switch, button, joystick, or the like. A left arrow is illustrated in FIG. 3. Thus, the appropriate response from the subject in this scenario would be to indicate the left direction.

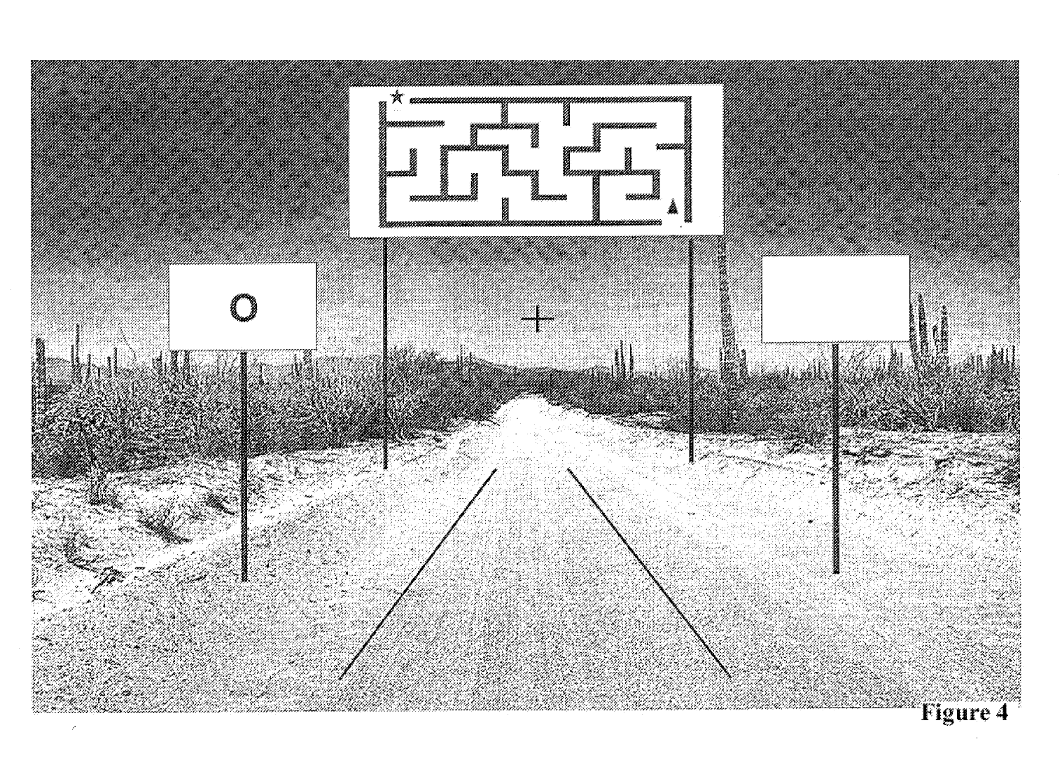

FIG. 4 is an exemplary illustration of the platform shown in FIG. 2, where a simple or choice reaction time information processing speed task is presented during the complex spatial navigation task, in accordance with the present invention. In this task, the subject's response is again based on what is displayed in the cognitive task display area. For example, the subject may be instructed to indicate a specific response on the feedback device when a letter appears (the “simple” task), or be instructed to indicate a specific response on the feedback device when a specific letter or letters appear, and indicate a different response when other letters are displayed in the cognitive task display area (the “choice” task). While the letter O is used in this exemplary illustration, numbers, symbols, shapes, colors, pictures, or the like could also be used.

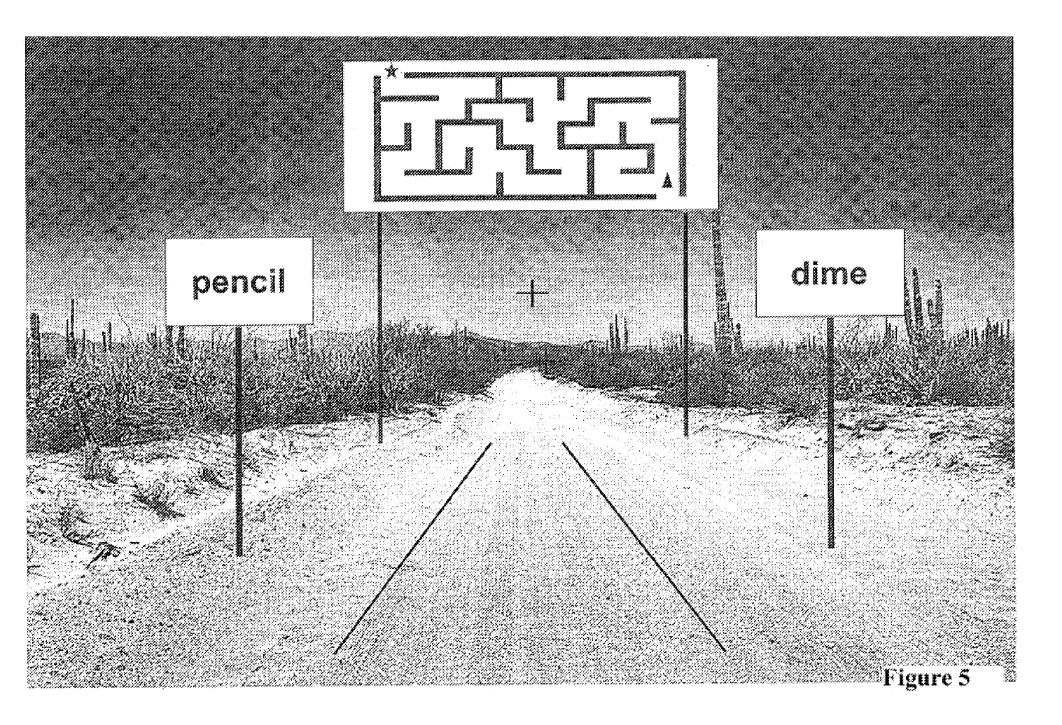

FIG. 5 is an exemplary illustration of the platform shown in FIG. 2, where a verbal-paired associates memory task is presented during the complex spatial navigation task, in accordance with the present invention. In this task, the subject's response is again based on what is displayed in the cognitive task display area. For example, each placard displays a noun to a subject during the spatial navigation task. The subject may be instructed to press one button if both nouns presented are living things, and another button if they are non-living objects. While living and non-living categories are used in this exemplary illustration, it should be noted that other categories, as well as other types of words or non-word stimuli, can be used. Further, the subject may be instructed to press one button if the pair of nouns presented matches a pair of nouns previously presented and another button if the nouns presented do not match a previously presented pair.

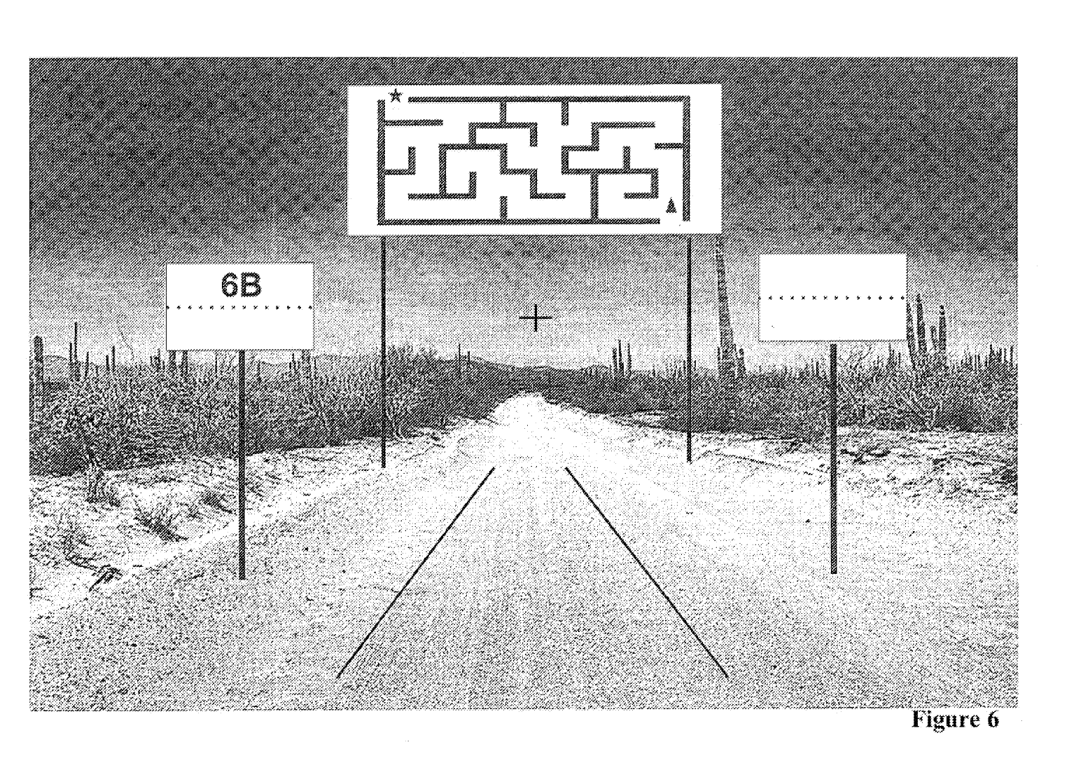

FIG. 6 is an exemplary illustration of the platform shown in FIG. 2, where an executive function number-letter switching task is presented during spatial navigation, in accordance with the present invention. In this task, the subject's response is again based on what is displayed in the cognitive task display area. For example, each of the placards is divided into an upper section and a lower section, and a combination of characters including a letter and a number are displayed together in one section of one of the placards. The subject is instructed to press one button if the number presented is even and a different button when the number is odd, when the combination of characters is presented in one of the two upper sections. The subject is also instructed to press one button if the letter presented is a vowel and a different button if the letter is a consonant, when the combination of characters is presented in one of the two lower sections.

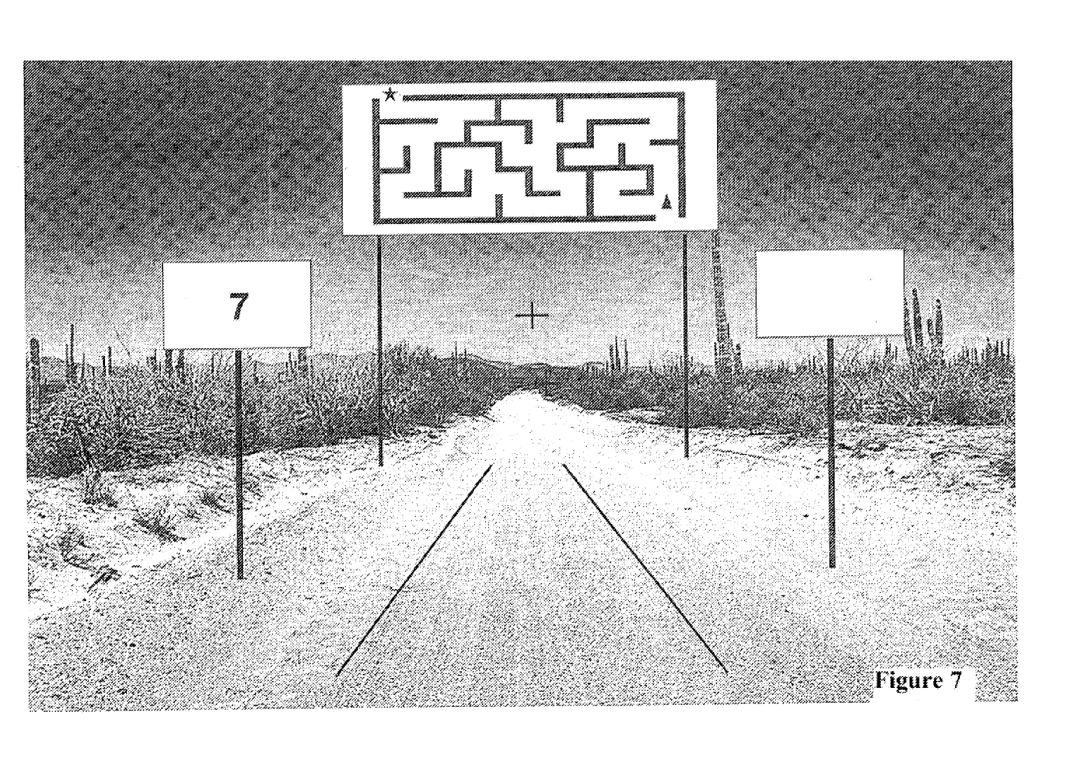

FIG. 7 is an exemplary illustration of the platform shown in FIG. 2, where an executive function updating n-back task is presented during the complex spatial navigation task, in accordance with the present invention. In this task, the subject is instructed to press one or more specific buttons when the integer presented matches a number presented n-back (e.g., 2-back) from the currently presented integer.

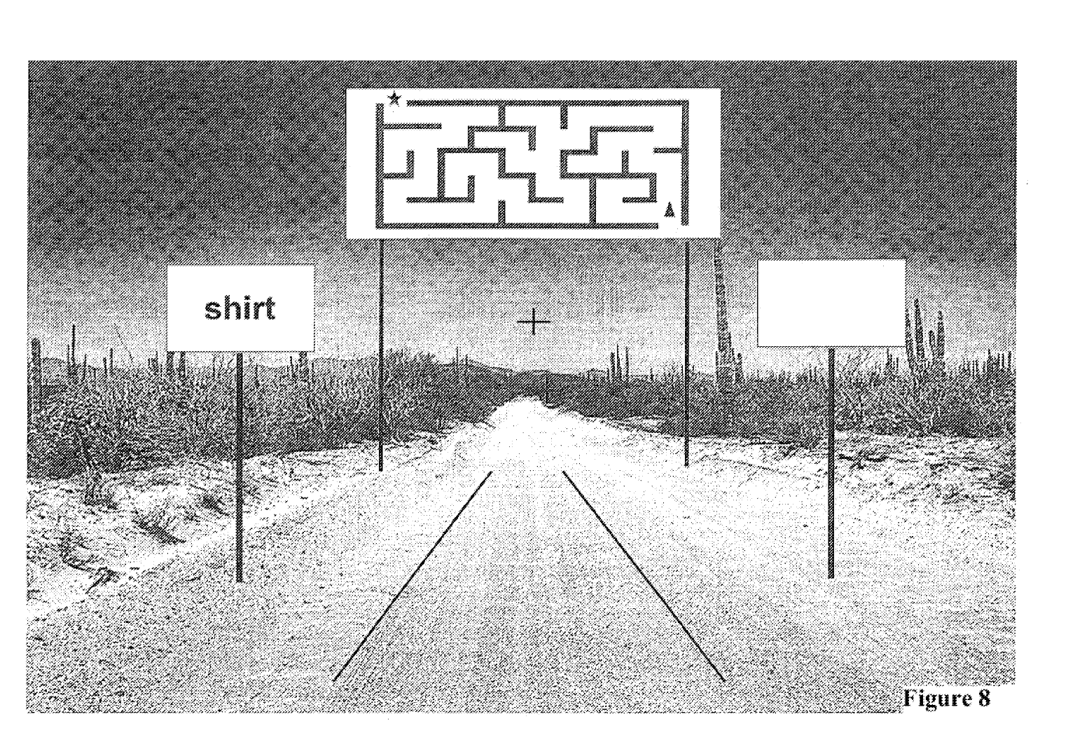

FIG. 8 is an exemplary illustration of the platform shown in FIG. 2, where a verbal list-learning memory task is presented during the complex spatial navigation task, in accordance with the present invention. In one portion of this task, the subject is instructed to press one or more specific buttons if the word presented fits a specific category, for example, if the word describes a non-living object. In another portion of this task, the subject is asked to press one button if the word presented was presented previously and another button if the word was not presented previously.

While FIGS. 3, 4, 5, 6, 7, and 8 illustrate exemplary cognitive tasks that may be displayed during the spatial navigation task, these exemplary cognitive tasks are non-limiting. It should be appreciated that any known cognitive task may be administered as part of the series of one or more additional cognitive tasks administered during the complex spatial navigation task. It should also be appreciated that these cognitive tasks may be administered without the subject being engaged in aerobic activity. For example, the subject may be stationary, or engaged in any activity at any level of exertion, including interval-type training that includes both aerobic and anaerobic activity.

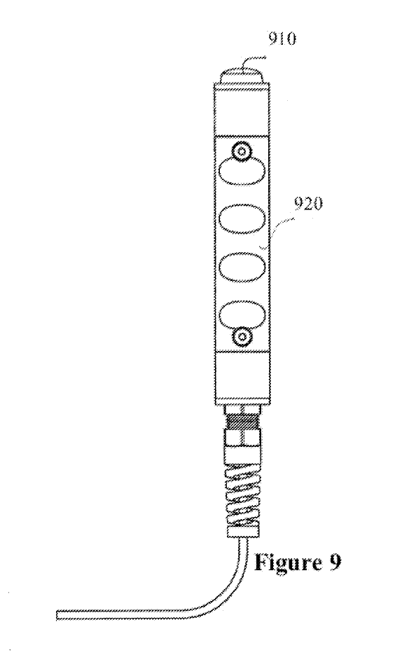

FIG. 9 is an exemplary feedback device in accordance with the present invention. At one end of the feedback device is a button 910 that is depressible to provide a response during one or more cognitive tasks. In some implementations, the button may be a two-position switch or a rocker switch. A pressure switch 920 is used to provide directional response to the complex spatial navigation task. In still other implementations, the feedback device may include only one switch, and the single switch may be used to indicate direction during the spatial navigation task and response during one or more cognitive tasks, where response is allocated based on the timing of the response relative to the task presented. Preferably, a subject is provided with a feedback device for each hand. The feedback device may be connected to the task administration system via a wired connection, such as a serial USB connection, or may be connected wirelessly via, for example Bluetooth or wi-fi.

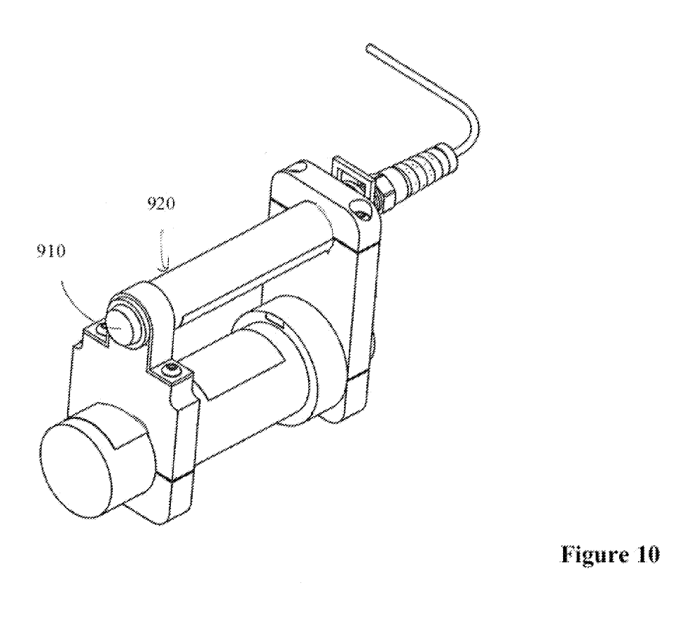

FIG. 10 illustrates exemplary positioning of the feedback device shown in FIG. 9 on the handlebar or grip of a recumbent stationary bicycle, in accordance with the present invention.

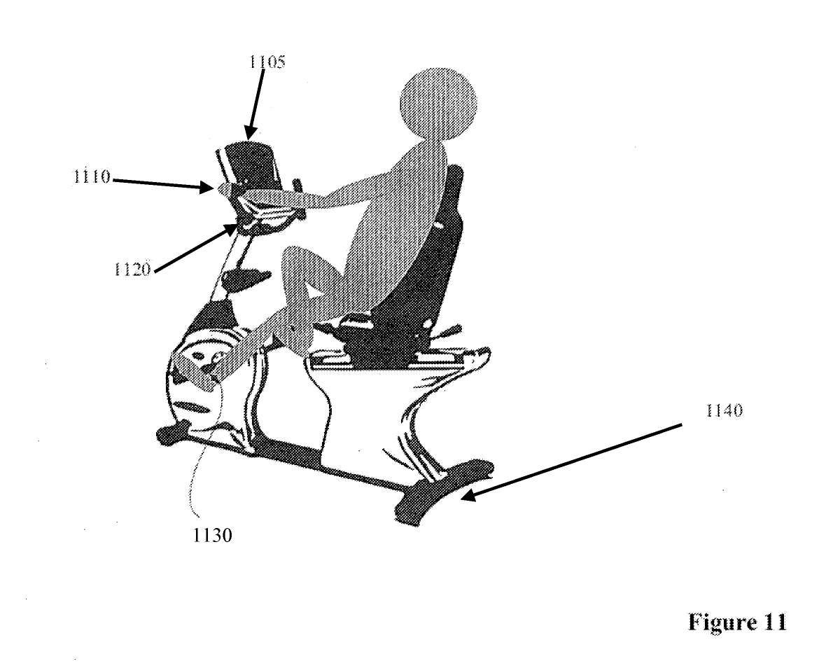

FIG. 11 illustrates an exemplary implementation of the present invention, using a recumbent stationary bicycle. The complex spatial navigation task and any additional cognitive tasks are displayed on display 1105 which may be, for example, a tablet computing device. Input devices 1110 and 1120 provide for subject response to the complex spatial navigation task and any additional cognitive tasks administered. Sensor 1130 monitors subject movement using, for example, an accelerometer while the subject engages in physical activity on exercise equipment such as recumbent stationary bicycle 1140.

In an exemplary implementation, an aerobic and cognitive training system test battery includes 5 modules lasting a total of 30 minutes of physical activity for the subject. Preferably, the subject is monitored and maintains an average heart rate between 60% and 80% of age-adjusted maximum heart rate during the battery in order to maximize the potential for cognitive neuroplastic benefits.

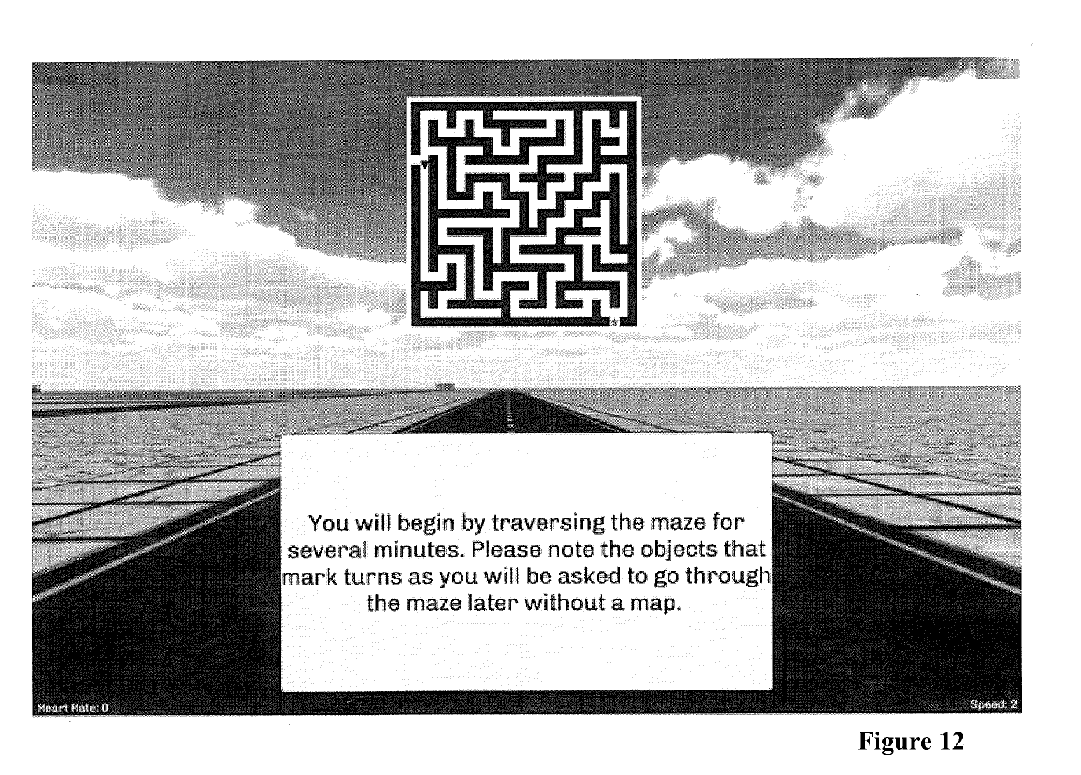

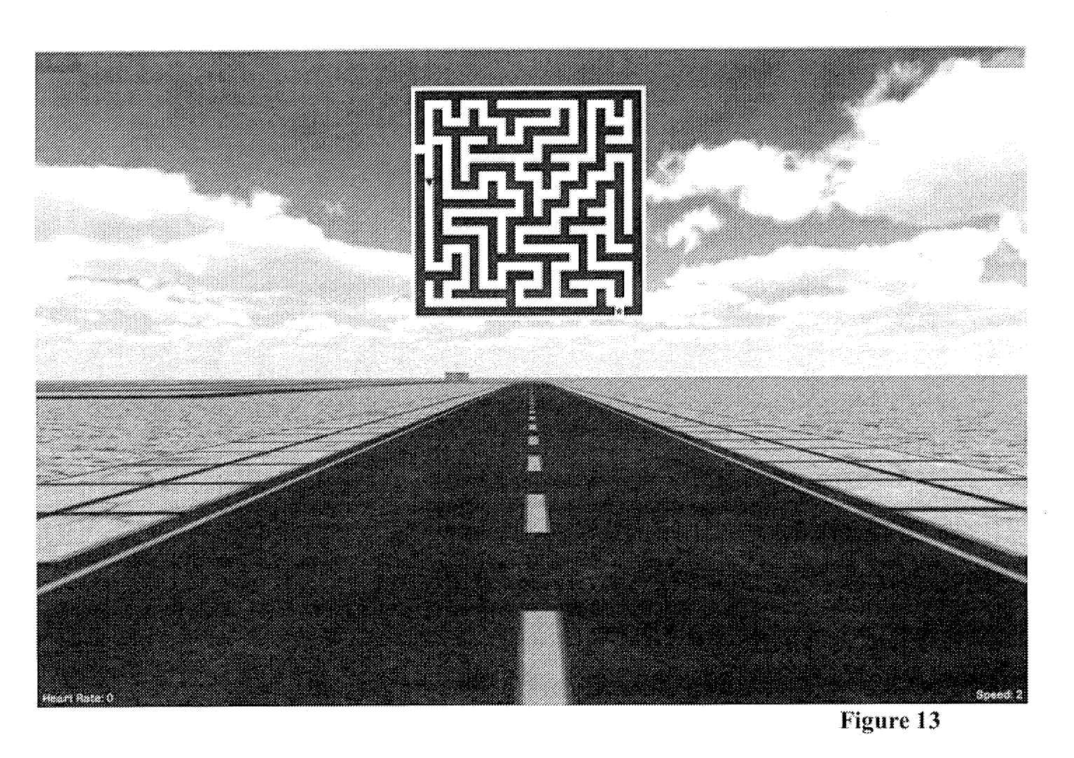

In Module 1, the complex spatial navigation task is presented to the subject. Specifically, the subject is instructed to navigate a maze presented via a display, and to learn the route through the maze with cues or landmarks presented visually in order to facilitate recall of correct turns to reach the end of the maze. A maze map is provided as a visual reference for the subject. The subject is instructed to remain in the center of the path indicated by a center target, much like staying in one's lane on a road, and to turn to navigate the maze to reach the end. The subject's position in the maze is followed and updated in real time on the maze map, and visual cues are presented to the subject for later recall of the route with or without use of the maze map guide. Preferably, instructions are presented for a period of time, such as 20 seconds, and can be advanced by a button press to begin the task. The difficulty/complexity of the complex spatial navigation task may be varied to suit the subject. Exemplary screenshots for the tasks described in Module 1 are provided in FIGS. 12 and 13.

For all complex spatial navigation tasks described herein, performance is assessed by the number of correct turns as a percent of total turns, time to reach the end of the maze/task, number of errors in going outside of center path guidelines, time on the solution path, time in the center of the path, distance traveled, solution distance traveled, and number of completed mazes as this complex spatial navigation task is completed.

In Module 2, the complex spatial navigation task from Module 1 is repeated, and a selected list learning or paired associates task is administered before, during, and/or after administration of the complex spatial navigation task. As in Module 1, instructions are presented to the subject for a period of time, such as 20 seconds, and the subject can begin the task itself by pressing a button to advance beyond the instructions. The cognitive tasks administered during the complex spatial navigation task of Module 2 include, but are not limited to, a verbal list-learning task, a non-verbal list learning task, a verbal paired-associates task, and a face-name paired associates task. Each of these cognitive tasks is scalable, for example, from 6-15 items or pairs.

In the verbal list-learning task, single words are presented to the subject, one at a time, on placards or displays to the left and right of the complex spatial navigation task display. Each word is presented for a prescribed time, such as 3 seconds, and is followed by an interstimulus interval (ISI) of, for example, 1 second. The subject is instructed to press the right button if the word describes a common object larger than a shoebox (e.g., truck), and to press the left button if the word describes a common object smaller than a shoebox (e.g., pencil). It should be noted that other categorical designations may be substituted (e.g., living vs. non-living). After all words are presented, a forced choice response task is presented to the subject, in which two words are presented simultaneously, and the subject is instructed to select the button that corresponds to the word that matches a word previously presented in the set. This list-learning task may be repeated.

In the non-verbal list-learning task, non-nameable closed figures composed of curved lines and angles are presented to the subject, one at a time, on the left or right placard or display. Each figure is presented for a period of time, such as 3 seconds, followed by an ISI of, for example, 1 second. The subject is instructed to press the right button if the figure has, for example, 3 or more angles, and the left button if the figure has, for example, fewer than three angles. After all of the figures are presented, a forced choice response task is presented to the subject, and two figures are presented simultaneously. The subject is instructed to press the button that corresponds to the figure that was presented previously. This list-learning task may be repeated.

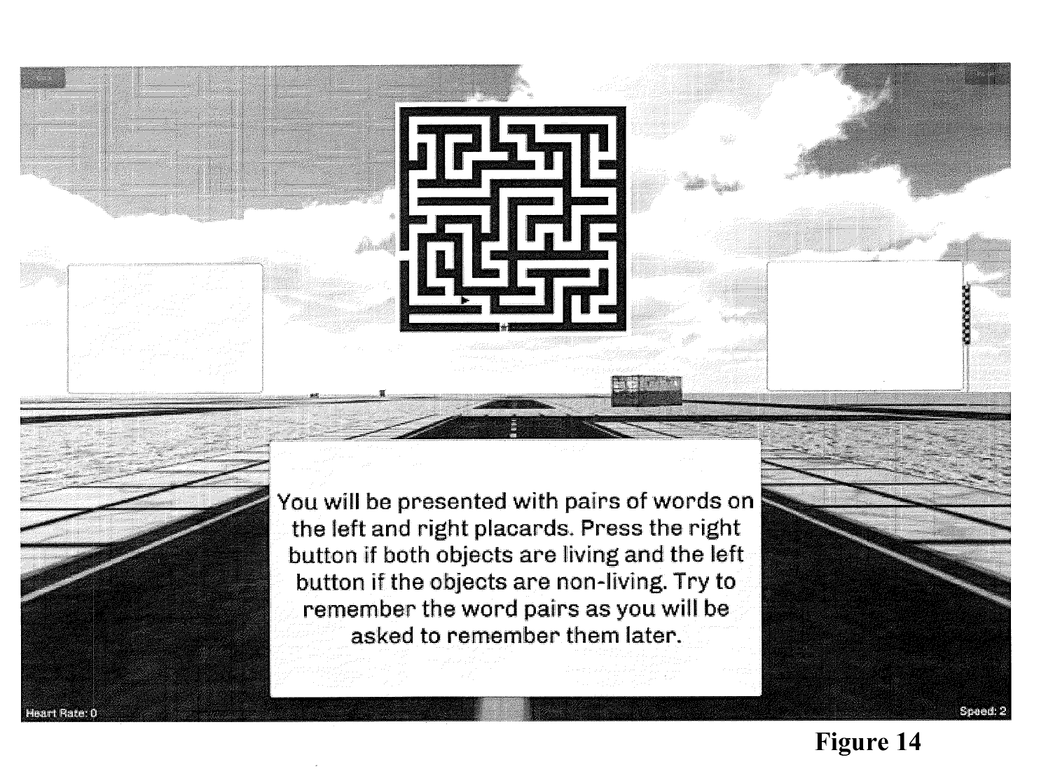

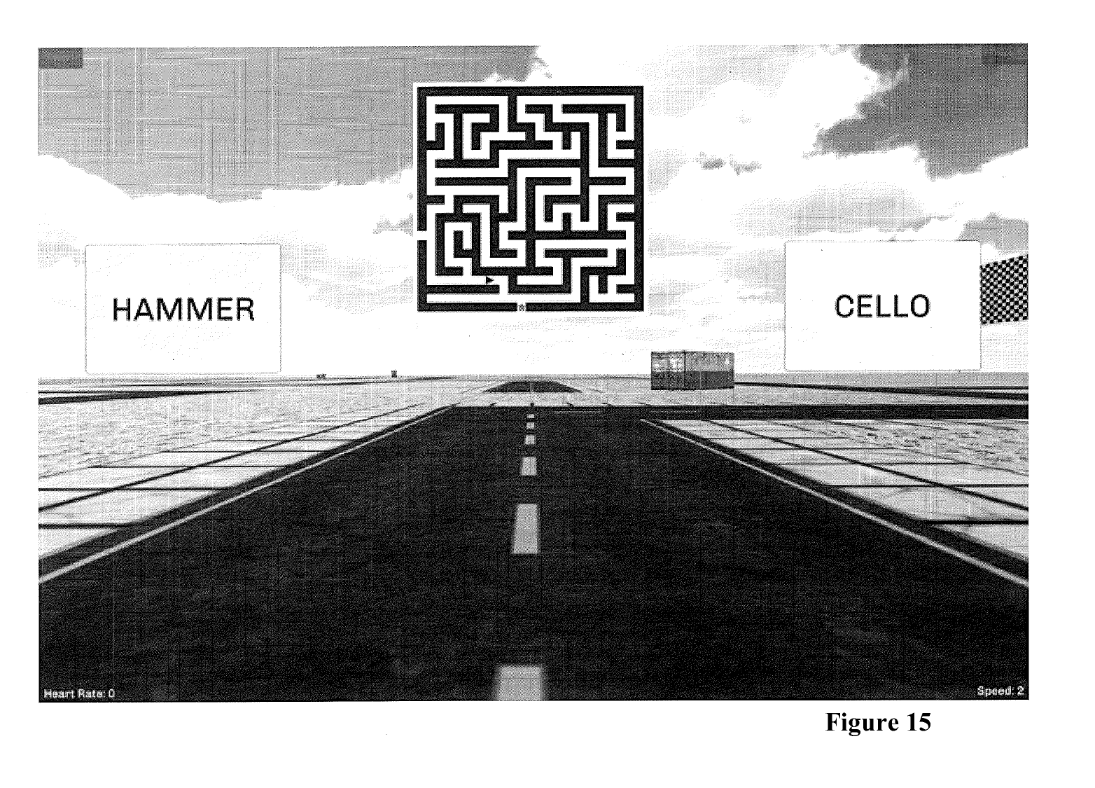

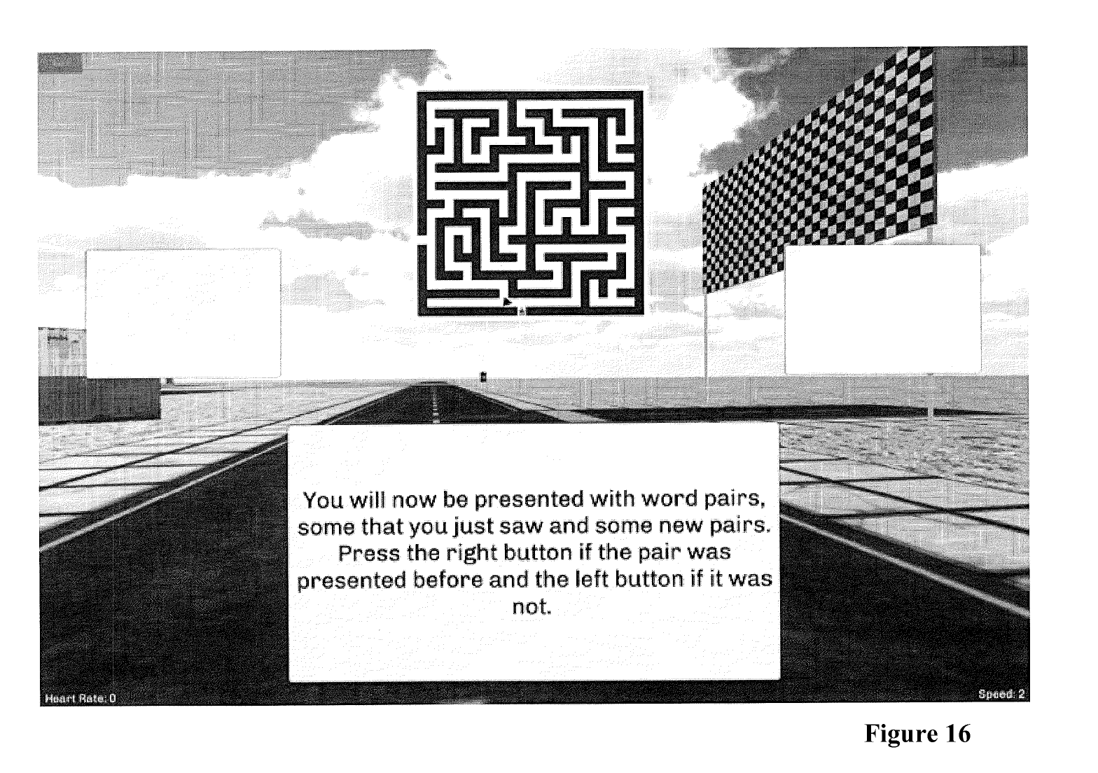

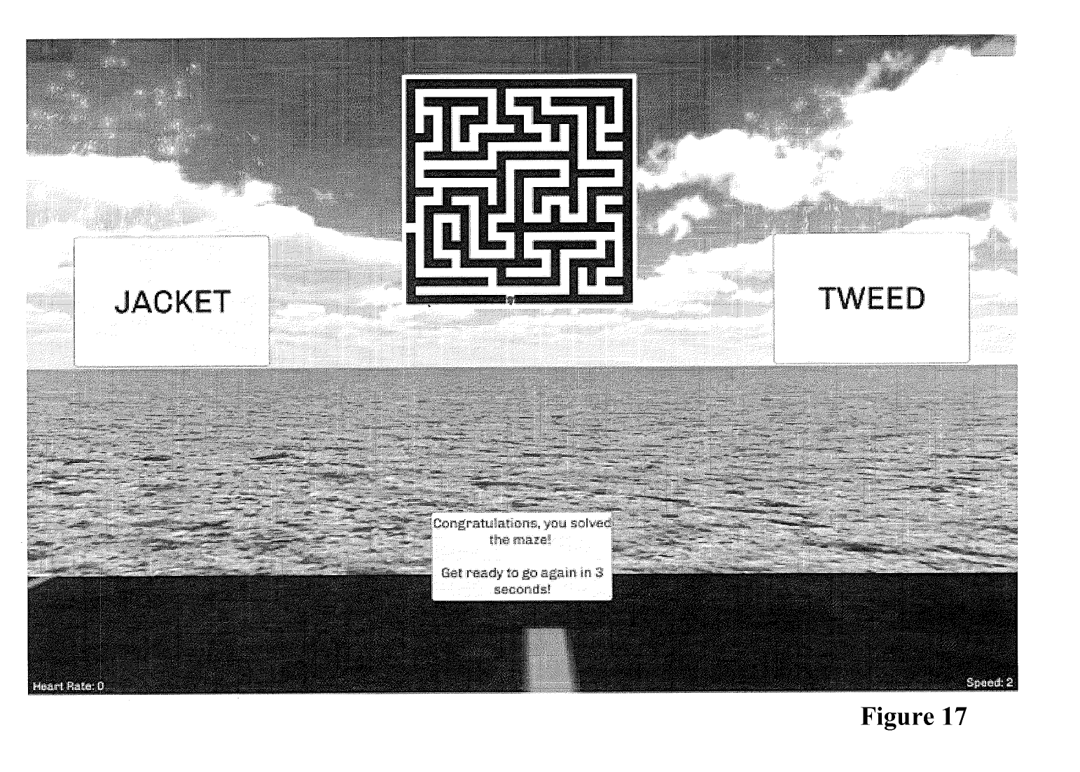

In the verbal paired-associates task, word pairs are presented simultaneously on the left and right placards or displays. Each word pair is presented for a prescribed period of time, such as 3 seconds, followed by an ISI of, for example, 1 second. The subject is instructed to press the right button if the two words in the pair are both living things (e.g., lion—tree) and the left button if they are non-living things (e.g., pencil—dime). It should be noted that other categorical designations may be substituted (e.g., bigger than a shoebox vs. smaller than a shoebox). After all of the word pairs are presented, sets of new and previously presented word pairs are shown with the task of pressing the right button if the word pair matches a word pair previously presented, and the left button if the pair was not presented previously. This paired associates task may be repeated. Exemplary screenshots for the verbal paired-associates described in Module 2 are provided in FIGS. 14-17.

In the face-name paired associates task, novel faces are presented to the subject on the left placard or display, and a first name is simultaneously presented on the right placard or display. Each face-name pair is presented for a prescribed period of time, such as 3 seconds, followed by an ISI of, for example, 1 second. The subject is instructed to press the right button if the face-name pair appears as a male, and the left button if the face-name pair appears as a female. After all face-name pairs are presented, new and previously presented face-name pairs are presented to the subject with the task of pressing the right button if the pair presented matches a pair previously presented in the set, and the left button if the pair was not presented previously. This paired associates task may be repeated.

It should be appreciated that, in Module 2, the set size and timing can be modified to increase or decrease the difficulty of the task. Accuracy and response time are measured, as is recognition, for the list-learning and paired associates tasks. Further, it should be appreciated that additional recognition tasks may be presented in later modules to assess delayed recognition of the learned items or pairs.

In Module 3, the complex spatial navigation task of Module 1 is repeated, with the addition of one or more selected executive function task that requires, for example, inhibition, switching, or updating/monitoring before, during, and/or after administration of the complex spatial navigation task. As with the other modules, instructions are presented for a period of time, and can be advanced by button press to begin the task.

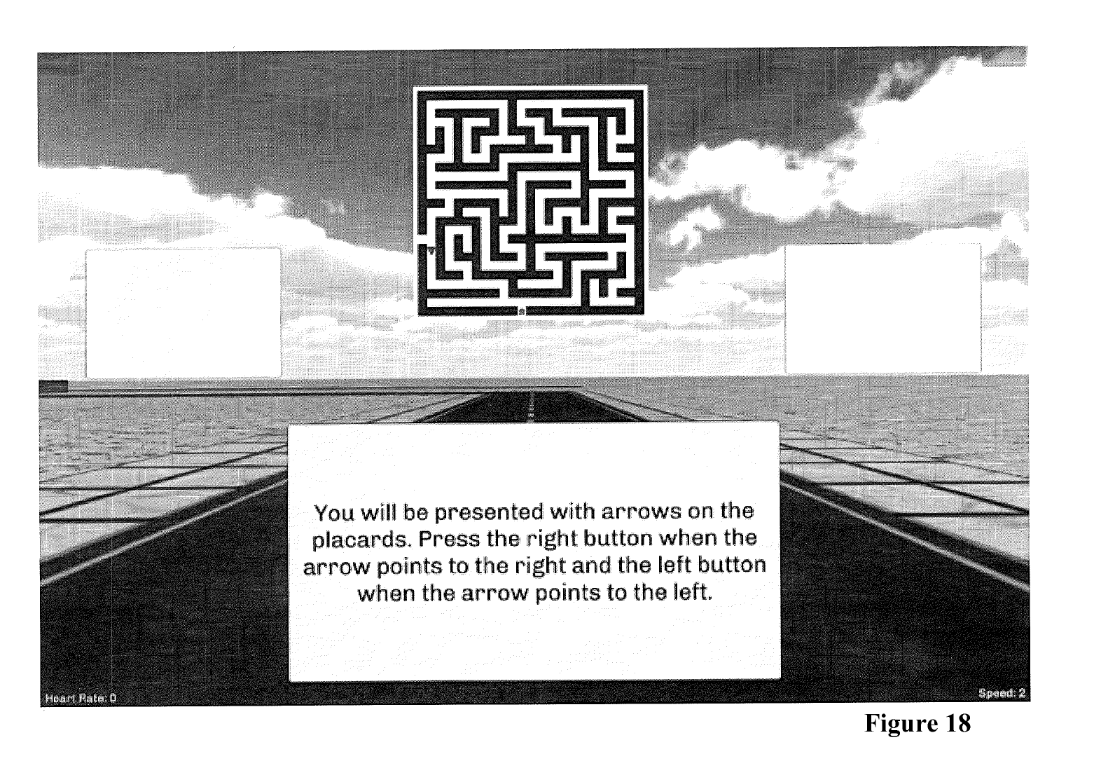

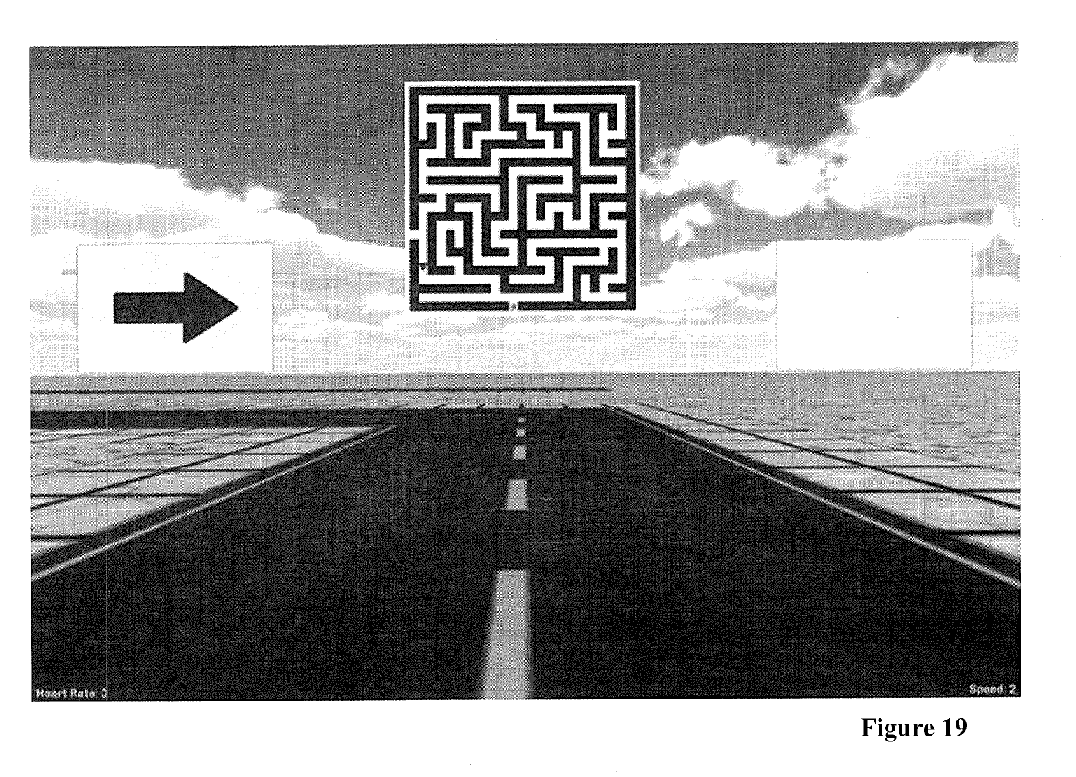

In the inhibition task, a Simon Task is used in which left and right pointing arrows are presented sequentially on the left or right placards/displays. The subject is instructed to press the right button when the arrow presented points to the right, and the left button when the arrow presented points to the left. Inhibition effects are imposed when a right pointing arrow is presented on the left side, and when the left pointing arrow is presented on the right side. The stimuli are presented for a prescribed period of time, such as 3 seconds, followed by, for example, randomly selected variable ISIs of, for example, 1, 2, and 4 seconds. Preferably, equal numbers of left and right pointing arrows are presented on the right and left sides. Accuracy and response time are the outcome measures for the response button presses, with inhibition time differences assessed between arrow location presentations. ISIs may or may not be variable in different implementations of this task. FIGS. 18 and 19 provide exemplary screenshots of inhibition tasks in accordance with Module 3.

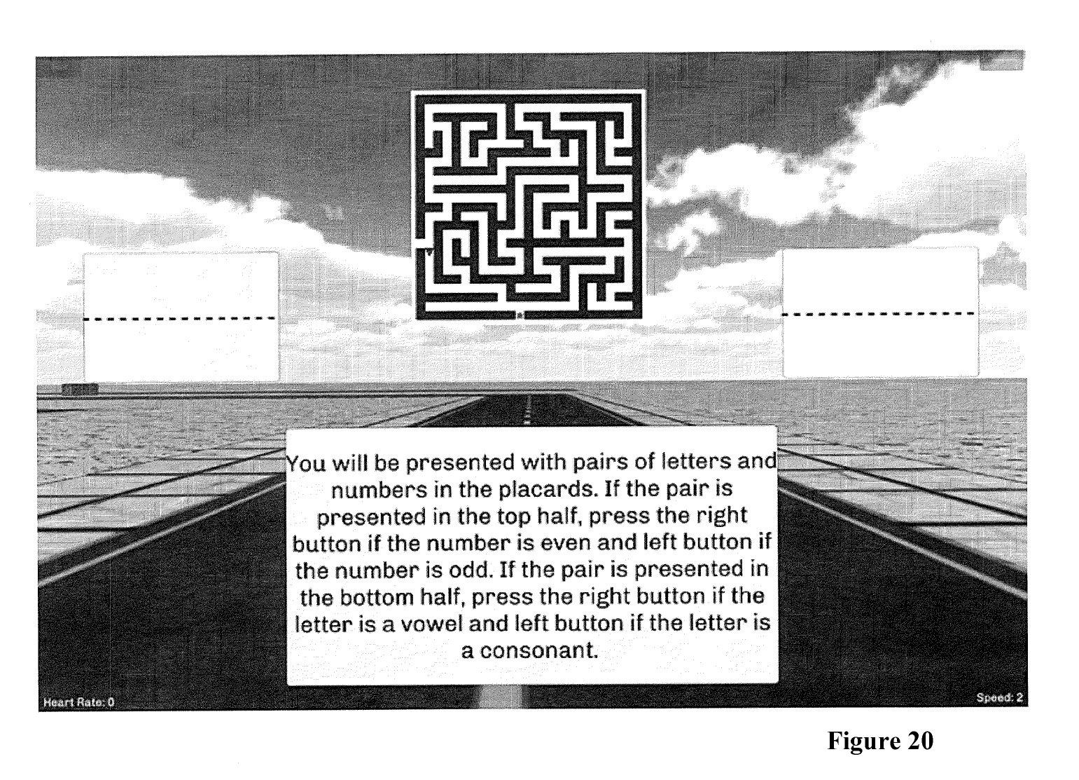

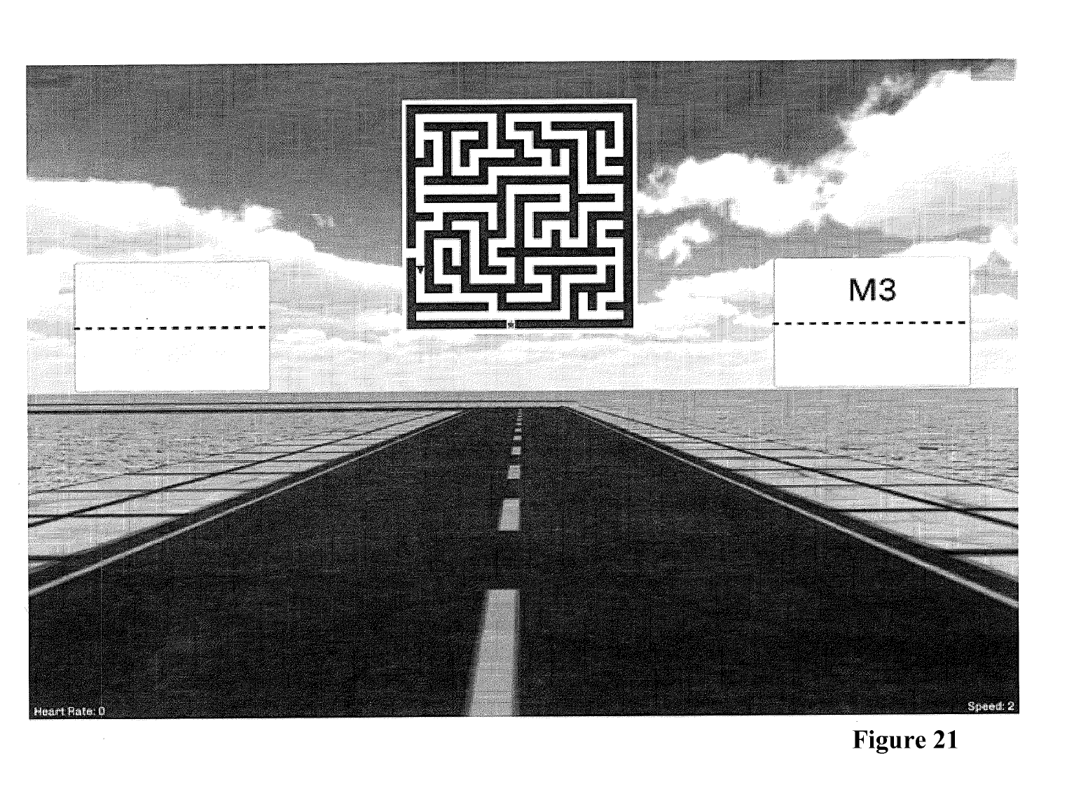

In the switching task, a number-letter task is presented to the subject. A number-letter pair (e.g., 6B) is presented in one of four quadrants from the top and bottom halves of the left and right placards/displays. When the stimuli is presented in the top half on the left or right side, the subject is instructed to press the right button if the number is even and the left button if the number is odd. When the number-letter pair is presented in the bottom halves of the left or right placards, the subject is instructed to press the right button if the letter is a vowel and the left button if the letter is a consonant. At least three blocks of stimuli are presented, with one block being presented in the top halves, one block being presented in the bottom halves, and a third block shifting, for example in a clockwise manner, with half the stimuli being presented on top and half being presented on the bottom. The stimuli are presented for a prescribed period of time, for example 3 seconds, followed by an ISI of, for example, 1 second. Accuracy and response time are measured, as well as shift costs in the time assessed between blocks. FIGS. 20 and 21 provide exemplary screenshots of switching tasks in accordance with Module 3.

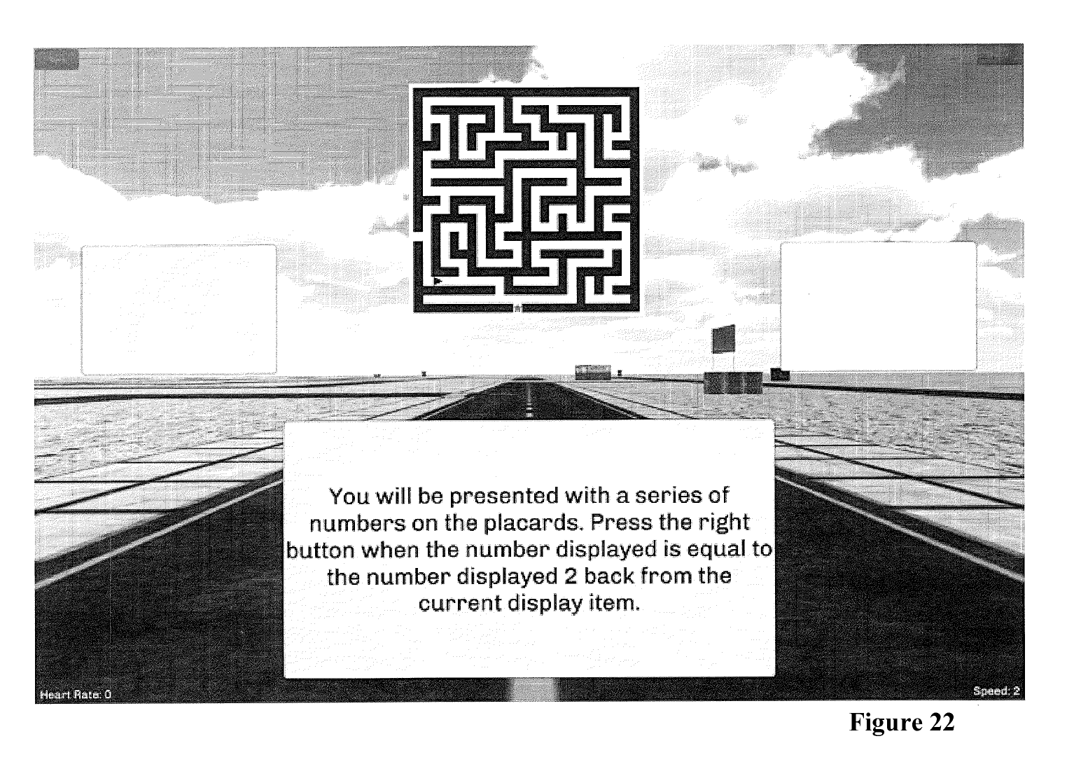

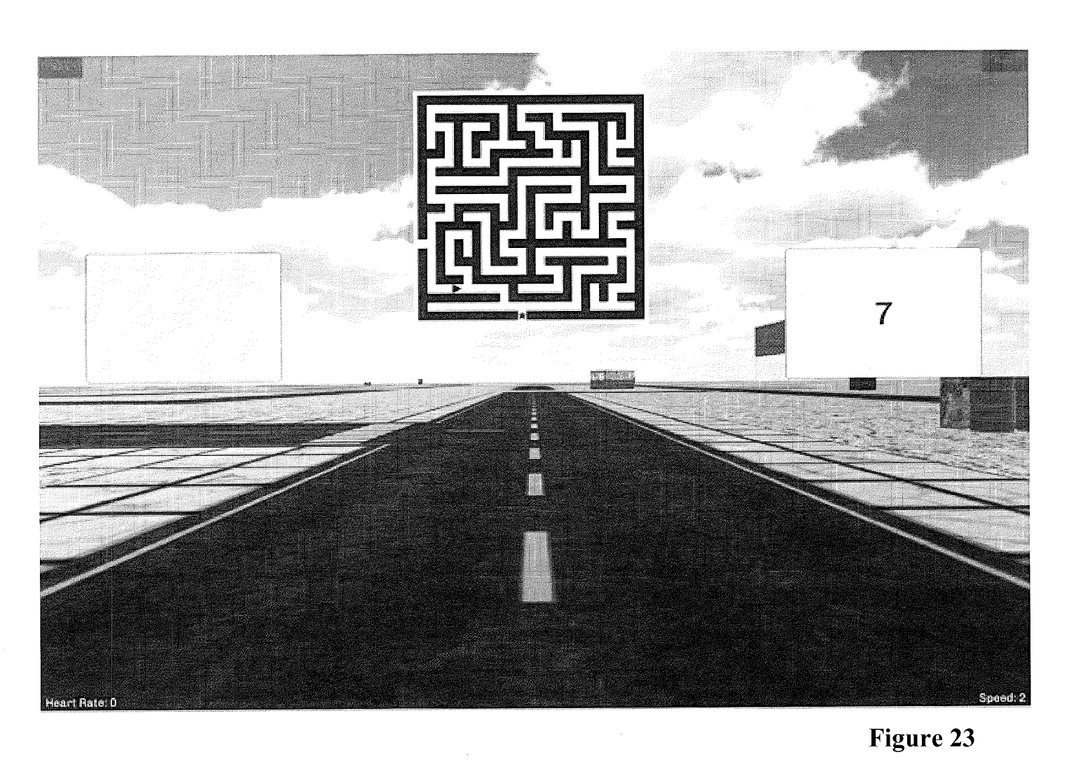

In the updating/monitoring task, an n-back task is used. A string of integers from 1 to 9 is presented to the subject sequentially on either the right or left side placards/displays. The subject is instructed to press both buttons when a number matches a number that was presented n-back from the current integer in the string. The set size can be adjusted to vary difficulty, as can the n-back condition. Accuracy and response time are measured, including omissions and commissions. FIGS. 22 and 23 provide exemplary screenshots of an updating/monitoring task in accordance with Module 3.

In Module 4, the complex spatial navigation task of Module 1 is repeated with the addition of simple and choice reaction time tasks before, during, and/or after administration of the complex spatial navigation task. Instructions are presented to the subject for a period of time, for example, 20 seconds, and can be advanced by a button press to begin the task.

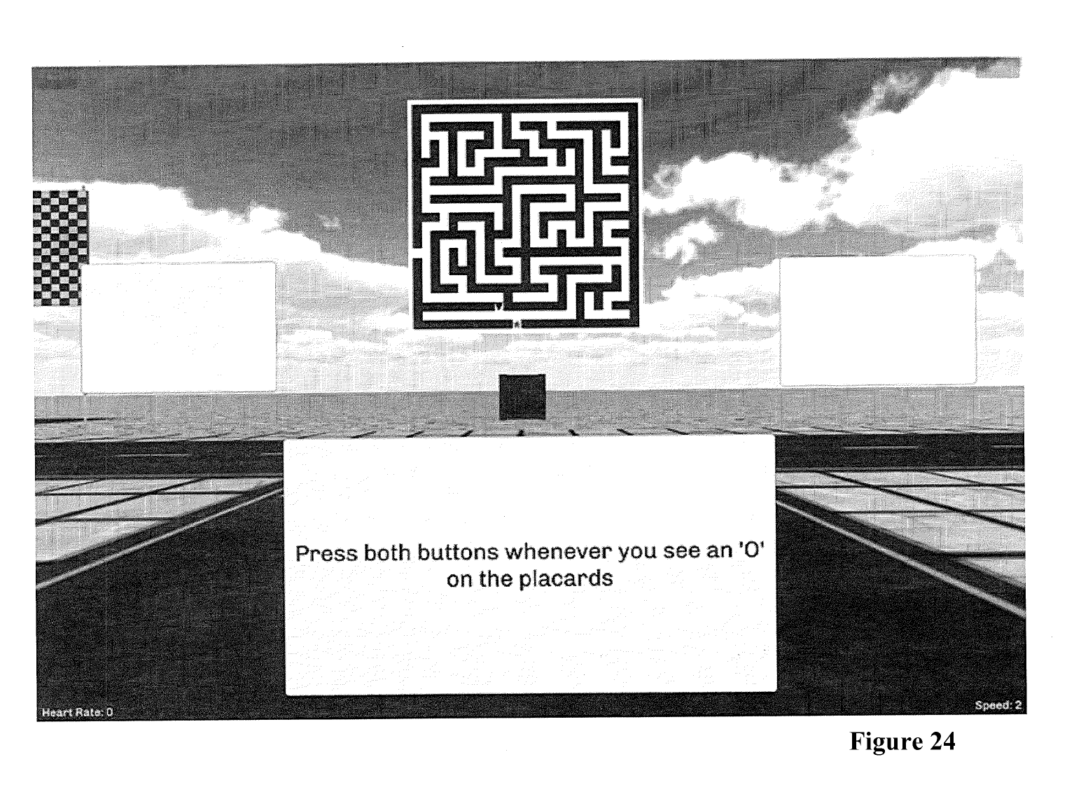

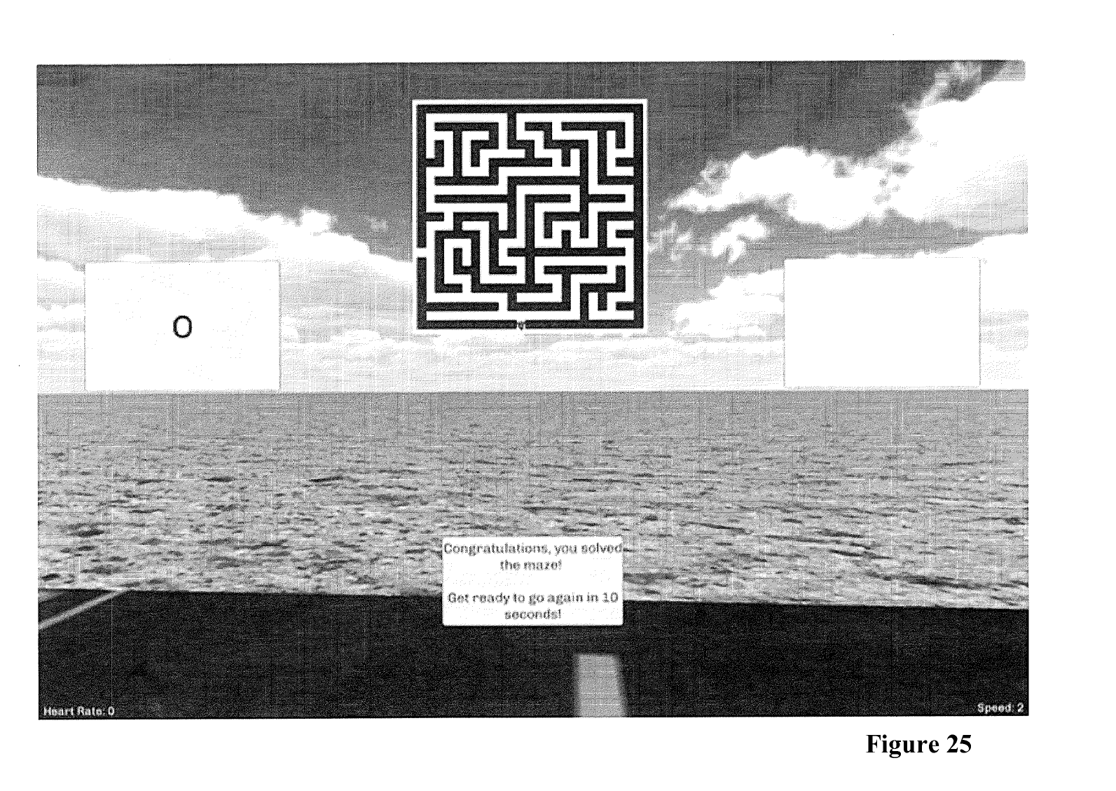

In the simple task, an “0” is randomly presented on the left or right placards/displays with, for example, randomly selected ISIs of 1, 2, or 4 seconds, so the target cannot be anticipated. The subject is instructed to press both buttons when the target is observed. Performance is measured by accuracy and response time. FIGS. 24 and 25 provide exemplary screenshots of the simple task in accordance with Module 4.

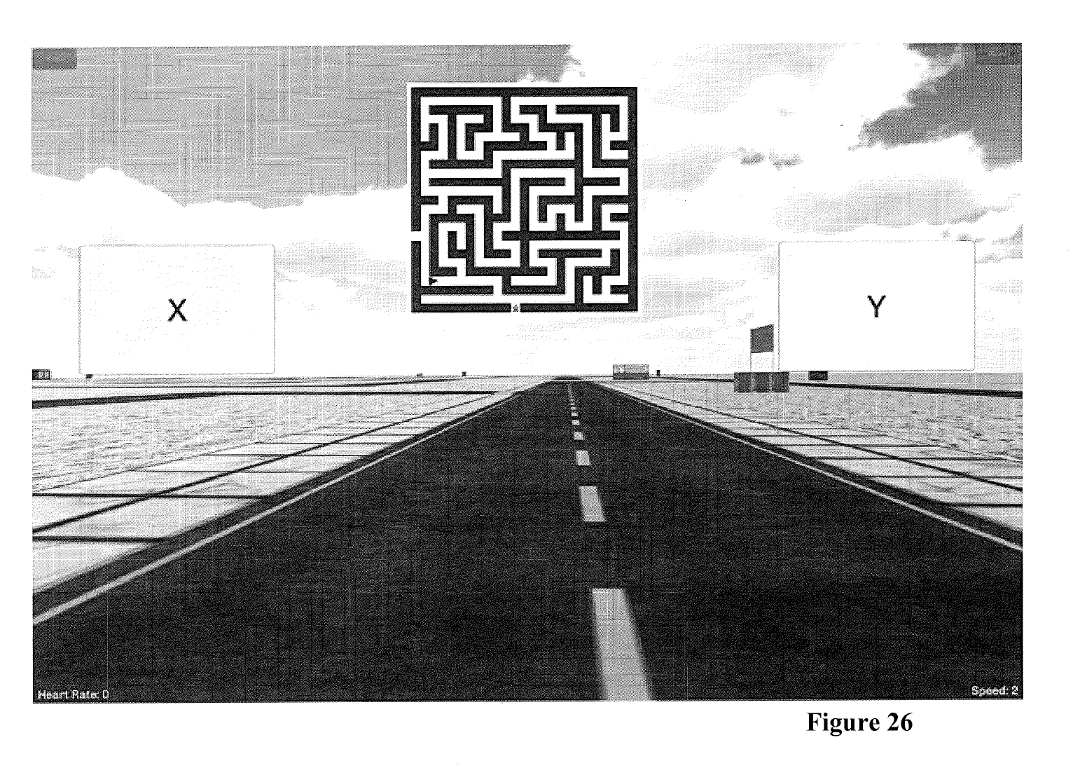

In the choice task, pairs of letters are presented, with one letter on each of the left and right placards/displays. The subject is instructed to press the right button when the letters are the same, and the left button when the letters in the pairs are different. Pairs are presented for a prescribed period of time, for example 3 seconds, followed by an ISI of, for example, 1 second. This may be varied. FIG. 26 provides an exemplary screenshot of the choice task in accordance with Module 4.

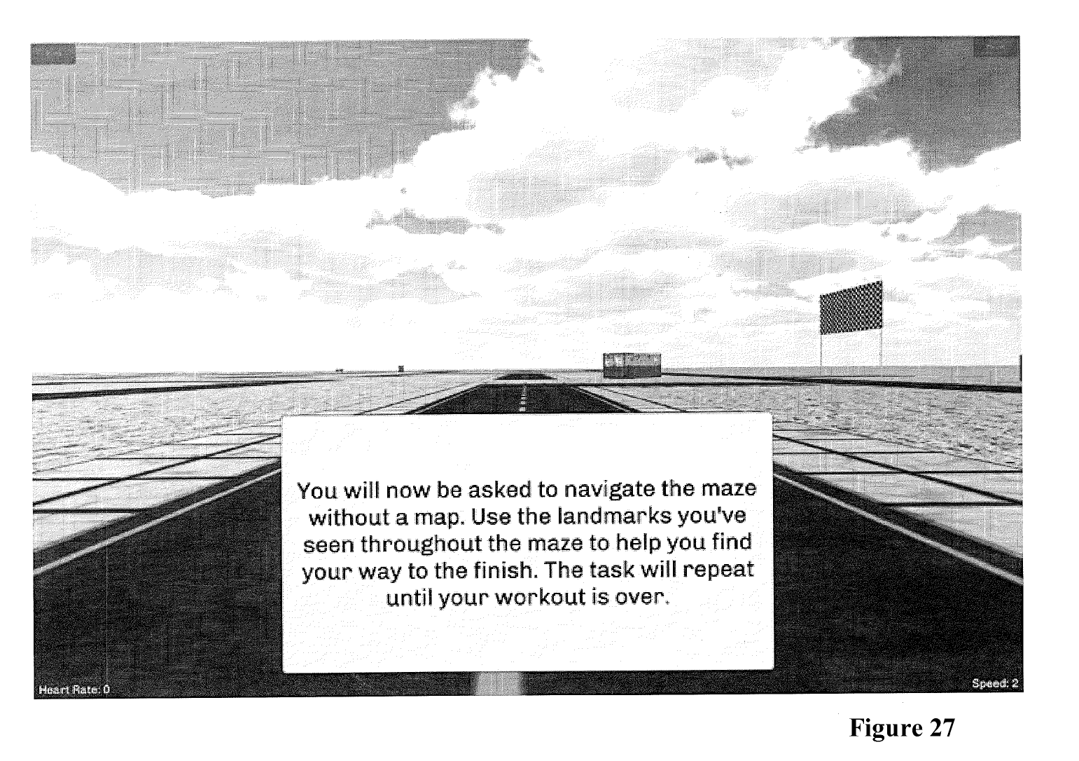

In Module 5, the complex spatial navigation task of Module 1 is repeated without the presentation of a maze map. The subject is instructed to navigate the same route based on the learned association of the visual cues and correct turns from the previously presented spatial navigation tasks while remaining in the center of the path. FIG. 27 provides an exemplary screenshot of the complex spatial navigation task in accordance with Module 5.

While the exemplary battery presented herein includes 5 modules, it should be appreciated that additional modules may be presented, or fewer modules may be presented without departing from the scope of the invention. Further, it should be appreciated that the modules or tasks within modules may be presented in any order without departing from the scope of the invention. Similarly, other cognitive tasks or assessments may be substituted for those described herein without departing from the scope of the invention.

What is claimed is:

1. A method for cognitive training, comprising:
administering a complex spatial navigation task to a subject.

2. A method for cognitive training according to claim 1, further comprising:
administering the complex spatial navigation task to the subject while the subject is engaged in physical activity.

3. A method for cognitive training according to claim 2, further comprising:
monitoring the subject's physiological response to the cognitive training.

4. A method for cognitive training according to claim 3, wherein the subject's progression through the complex spatial navigation task is based upon the subject's physiological response to the cognitive training.

5. A method according to claim 4, wherein an increase in physiological response increases the speed at which the subject progresses through the complex spatial navigation task, and a decrease in physiological response decreases the speed at which the subject progresses through the complex spatial navigation task.

6. A method according to claim 1, further comprising:
administering, during the complex spatial navigation task, a cognitive task selected from a group consisting of cognitive tasks that test memory, executive functions, information processing speed, language processing, and visuospatial/visuoperceptual functions.

7. A method according to claim 6, further comprising:
administering the complex spatial navigation task and the cognitive task while the subject is engaged in physical activity.

8. A method according to claim 7, wherein the physical activity is sufficient to cause a subject to reach a target aerobic intensity range.

9. A method for cognitive training, comprising:
administering a complex spatial navigation task to a subject;
administering the complex spatial navigation task to the subject a second time, wherein during the second administration of the complex spatial navigation task, a cognitive task selected from the group of cognitive tasks that test memory, executive functions, information processing speed, language processing, and visuospatial/visuoperceptual functions is simultaneously administered to the subject.

10. A method according to claim 9, wherein the complex spatial navigation task and the cognitive tasks is administered while the subject is engaged in physical activity.

11. A method according to numbered claim 10, further comprising determining the accuracy of subject response to the spatial navigation task and the cognitive task, and providing feedback to the subject regarding performance.

12. A method according to claim 9, further comprising repeating administration of the complex spatial navigation task while simultaneously administering a second cognitive task selected from the group of cognitive tasks that test memory, executive functions, information processing speed, language processing, and visuospatial/visuoperceptual functions is simultaneously administered to the subject.

13. A method according to claim 12, further comprising, for each task administered to the subject, measuring the subject's performance and providing feedback regarding performance.

14. A system for aerobic and cognitive training, comprising:
a sensor for monitoring subject response to physical activity;
a first input device for providing subject to a complex spatial navigation activity;
a display device; and
a computer including a non-transitory computer readable storage medium storing software configured to administer a spatial navigation task to a subject on the display device, to receive responses to the complex spatial navigation task from the first input device, wherein the software determines at least one performance metric for the complex spatial navigation administered, stores the performance metric, and provides feedback to the subject regarding subject's performance, and wherein the software is configured to monitor data collected from the sensor regarding subject's physical response to activity, and prompt the subject to stay within a prescribed level of exertion.

15. A system in accordance with claim 14, wherein the prescribed level of exertion is a target heart rate range, and the sensor for monitoring subject response to a physical activity is a heart rate monitor.

16. A system in accordance with claim 14, further comprising a subject activity sensor, where the software monitors the subject activity sensor, and provides feedback to the subject regarding the level of activity detected.

17. A system in accordance with claim 16, wherein the subject activity sensor is an accelerometer.

18. A system in accordance with claim 14, wherein the software is configured to control a subject's progress through the complex spatial navigation task based on data received from the sensor for monitoring subject response to physical activity.

19. A system in accordance with claim 18, wherein the subject's speed of progress through the complex spatial navigation task increases as the sensor indicates an increase in level of exertion, and the subject's speed of progress through the spatial navigation task decreases as the sensor indicates a decrease in level of exertion.

20. A system in accordance with claim 16, further comprising:
a second input device for providing subject response one or more cognitive tasks,
wherein the software is further configured to administer the one or more cognitive tasks to the subject before, during, and/or after the course of the complex spatial navigation task, to receive responses to the one or more cognitive tasks, determine at least one performance metric based on the received responses, store the performance metric, and provide feedback regarding the subject's performance.
